(19) United States
(12) Patent Application Publication
(10) Pub. No.: US 2024/0390395 A1
(43) Pub. Date: Nov. 28, 2024

(54) APPLICATION OF ION CHANNEL BLOCKER IN TREATMENT AND/OR PREVENTION OF HEPATIC FIBROSIS

(71) Applicant: Affiliated Hospital of Zunyi Medical University, Zunyi (CN)

(72) Inventors: Jingyu Xu, Zunyi (CN); Rui Xie, Zunyi (CN); Qian Du, Zunyi (CN); Yanxia Hu, Zunyi (CN); Jianhong Ding, Zunyi (CN); Xianli Wu, Zunyi (CN); Xiaoxu Yang, Zunyi (CN); Jun Lou, Zunyi (CN); Qi Liu, Zunyi (CN); Zhuo Li, Zunyi (CN)

(21) Appl. No.: 18/467,687

(22) Filed: Sep. 14, 2023

(30) Foreign Application Priority Data
May 22, 2023 (CN) 2023105795521

(51) Int. Cl. A61K 31/64; A61P 1/16

(52) U.S. Cl. CPC A61K 31/64; A61P 1/16

(57) ABSTRACT

An ion channel blocker for treatment and/or prevention of hepatic fibrosis is provided. Based on the fact that the activation of hepatic stellate cells is an important link in anti-hepatic fibrosis, it is found that gliclazide has the effect of inhibiting the activation of hepatic stellate cells by in vitro and in vivo experiments. Further exploration reveals that gliclazide mainly inhibits the activation of hepatic stellate cells by inhibiting autophagy pathways, and thus the gliclazide can be used to prepare medicaments for treatment and/or prevention of hepatic fibrosis.

### TECHNICAL FIELD

The disclosure relates to the technical field of ion channels and hepatic fibrosis, and particularly to an application/use of an ion channel blocker in treatment and/or prevention of hepatic fibrosis.

### BACKGROUND

Patients with chronic hepatic disease account for over 800 million people worldwide, including 2 million deaths annually due to various hepatic diseases. Hepatic fibrosis (HF) is not only the common pathological basis of all chronic hepatic diseases, but also a key link in the development of cirrhosis and hepatic cancer. Research has shown that hepatic fibrosis and even early cirrhosis can be reversed. However, current anti-fibrosis measures mainly focus on eliminating the causes of hepatic fibrosis, such as antiviral therapy, abstinence from alcohol, and dietary control, which generally have shortcomings such as long treatment courses, poor efficacy, multiple adverse reactions, and poor patient compliance. Therefore, people have been searching for a method that can significantly reverse hepatic fibrosis.

At present, it is believed that the activation of hepatic stellate cells (HSCs) and the excessive deposition of extracellular matrix (ECM) are core links in the pathophysiological changes of hepatic fibrosis. HSCs, originally identified by Carl von Kupffer in 1876, are localized in the subendothelial space of Disse, interposed between liver sinusoidal endothelial cells (LSECs) and hepatocytes, and the HSCs account for about 10% of all resident hepatocytes. In a normal liver, HSCs maintain a non-proliferative and stationary phenotype. After liver injury or in vitro culture, HSCs are activated, the activated HSCs transform from vitamin A-storing cells into myofibroblasts, with proliferation, contraction, inflammation, and chemotaxis, and are characterized by increased production of ECM. The activation of HSCs involves multiple regulatory mechanisms, including autophagy, endoplasmic reticulum stress, oxidative stress, retinol and cholesterol metabolism, epigenetics, and receptor-mediated signaling, which reveal the complexity of activation of the HSCs. At present, some targeting mechanisms of the activation of HSCs caused by the disorder of key molecular channels have been found, for example, transforming growth factor-beta (TGF-β) and platelet-derived growth factor (PDGF) participating in the activation of HSCs have been clarified. However, unfortunately, there is still a lack of anti-fibrosis therapy targeting HSCs in clinic. Therefore, exploring other channels leading to the activation of HSCs may lead to the discovery of more effective anti-fibrotic therapeutic targets.

Ion channels are a hot research field in recent years. Some ion channels are considered to play an important role in the occurrence, migration, invasion and metastasis of digestive tract cancers such as gastric cancer and hepatic cancer.

### SUMMARY

A purpose of the disclosure is to provide an application of an ion channel blocker in treatment and/or prevention of hepatic fibrosis.

In order to achieve the above purpose, the disclosure provides following technical solutions.

Specifically, an application of an ion channel blocker in treatment and/or prevention of hepatic fibrosis is provided by the disclosure.

In an embodiment, the ion channel blocker is a potassium ion channel blocker.

In an embodiment, the potassium ion channel blocker includes gliclazide.

In an embodiment, the hepatic fibrosis includes carbon tetrachloride (CCl₄)-induced hepatic fibrosis.

In the disclosure, a use of the gliclazide in preparing a medicament to inhibit autophagy of hepatic tissue is provided.

In the disclosure, a use of the gliclazide in preparing a medicament to inhibit proliferation of hepatic stellate cells (HSCs) is provided.

In an embodiment, the HSCs include rat hepatic stellate cells (HSCs-T6).

In the disclosure, a use of the glielazide in preparing a medicament to down-regulate alpha-smooth muscle actin (α-SMA) of hepatocytes is provided.

In the disclosure, a use of the gliclazide in preparing a medicament to down-regulate microtubule-associated protein 1A/1B-light chain 3 (LC3) of the hepatocytes is provided.

In the disclosure, a use of the gliclazide in preparing a medicament to up-regulate p62 proteins (also referred to as sequestosome 1) of the hepatocytes is provided.

The disclosure has the following technical effects. Based on the fact that the activation of the HSCs is an important link in anti-hepatic fibrosis, it is found that gliclazide has the effect of inhibiting the activation of the HSCs by in vitro and in vivo experiments. Further exploration reveals that gliclazide inhibits the activation of the HSCs mainly by inhibiting autophagy pathways, and thus the gliclazide can be used to prepare medicaments for treatment and/or prevention of hepatic fibrosis.

### BRIEF DESCRIPTION OF DRAWINGS

In order to illustrate embodiments of the disclosure or technical solutions in the related art more clearly, the attached drawings required in the embodiments will be briefly introduced below. Apparently, the attached drawings in the following description are only some embodiments of the disclosure. For those ssacrificed in the art, other attached drawings can be obtained based on these attached drawings without any creative work.

FIG. 1 illustrates degrees of fibrosis of hepatic tissues observed by hematoxylin-eosin (HE) staining and Sirius-red staining according to an embodiment 1.

FIGS. 2A-2B illustrate statistical diagrams of level changes of aspartate transaminase (AST) and alanine aminotransferase (ALT) detected in serum according to the embodiment 1.

FIGS. 3A-3B illustrate expression changes and statistical data of alpha-smooth muscle actin (α-SMA) in the hepatic tissues detected by immunohistochemistry according to an embodiment 2.

FIGS. 4A-4B illustrate expression changes and statistical data of a-SMA in the hepatic tissues detected by Western blotting according to an embodiment 3.

FIGS. 5A-5B illustrate expression changes and statistical data of microtubule-associated protein 1A/1B-light chain 3 (LC3) in the hepatic tissues detected by Western blotting according to the embodiment 3.

FIGS. 6A-6B illustrate expression changes and statistical data of p62 in the hepatic tissues detected by Western blotting according to the embodiment 3.

FIG. 7 illustrates a statistical diagram of effects of co-culture of gliclazide and transforming growth factor-betal (TGF-β1) on proliferation of hepatic stellate cells (HSCs) detected by cell counting kit-8 (CCK8) according to an embodiment 4.

FIGS. 8A-8B illustrate expression changes and statistical data of α-SMA in the HSCs detected by Western blotting according to an embodiment 5.

FIGS. 9A-9B illustrate expression changes and statistical data of LC3 in the HSCs detected by Western blotting according to the embodiment 5.

FIGS. 10A-10B illustrate expression changes and statistical data of p62 in the HSCs detected by Western blotting according to the embodiment 5.

FIG. 11A-11B illustrate expression changes and statistical data of α-SMA in HSCs after the addition of rapamycin detected by Western blotting according to the embodiment 5.

### DETAILED DESCRIPTION OF EMBODIMENTS

Various exemplary embodiments of the disclosure will be described in detail below. This detailed description should not be taken to be a limitation of the disclosure, but rather should be taken as a more detailed description of some aspects, features, and embodiments of the disclosure.

The technical methods of the disclosure are conventional in the art unless otherwise specified, and the reagents or raw materials used are purchased from commercial channels or have been published unless otherwise specified.

### Embodiment 1: Mouse Hepatic Fibrosis Induced by Carbon Tetrachloride (CCl₄)

Experimental materials include 60 healthy male C57 mice (20-25 g per mouse, purchased from Chongqing Tengxin Biotechnology Co., Ltd.), gavage needles, 1 milliliter (mL) syringes, CCI4 solution, olive oil, and gliclazide solution.

An experimental method is as follows. The CCl₄ solution is prepared by dissolving CCl₄ in the olive oil (a volume ratio of CCl₄ to olive oil is 1:4), and the prepared CCl₄ solution should be used immediately. The gliclazide solution is prepared by dissolving gliclazide in physiological saline, the concentration of the gliclazide solution is 500 micromoles per liter (μM), and the prepared gliclazide solution should be used immediately.

The mice are randomly divided into four groups including a, b, c, and d.
a. Normal control group.
b. Model group: mice are intragastrically administered with the CCl₄ solution, 0.1 mL/20 g (i.e., 0.1. mL CC14 per 20 g of body weight of mice), twice a week for 8 weeks.
c. CCl₄+gliclazide group: mice are intragastrically administered with the CCl₄ solution alone, 0.1 mL/20 g, twice a week for 2 weeks; then the mice are given with the gliclazide solution and the CCl₄ solution together for 6 weeks; and an administration method of the gliclazide solution is 0.2mL/20g and intraperitoneal injection once a day.
d. Gliclazide group: the gliclazide solution is intraperitoneally injected into mice for 8 weeks; and an administration method of the gliclazide solution is 0.2 mL/20 g and intraperitoneal injection once a day.

The mice are sacrificed at the 2^{nd}, 4^{th}, 6^{th} and 8^{th} weeks of modeling respectively, and one mouse in each group is sacrificed each time. Hepatic samples obtained from the sacrificed mice are taken out and stored in liquid nitrogen at −80 Celsius degrees (° C.), and the other hepatic samples are fixed in paraformaldehyde with a concentration of 4% for 24 hours, then the other hepatic samples are embedding in paraffin. The degrees of severity of hepatic fibrosis are observed by appearance, Sirius-red staining, and hematoxylin-eosin (HE) staining. If staining results indicate hepatic fibrosis, a model of mouse hepatic fibrosis is considered to be established successfully. Then all mice are sacrificed, and the eyeball blood is taken to measure hepatic function indexes including alanine aminotransferase (ALT) and aspartate transaminase (AST). In addition, the hepatic tissues are fixed with formaldehyde and embedded in paraffin for later use.

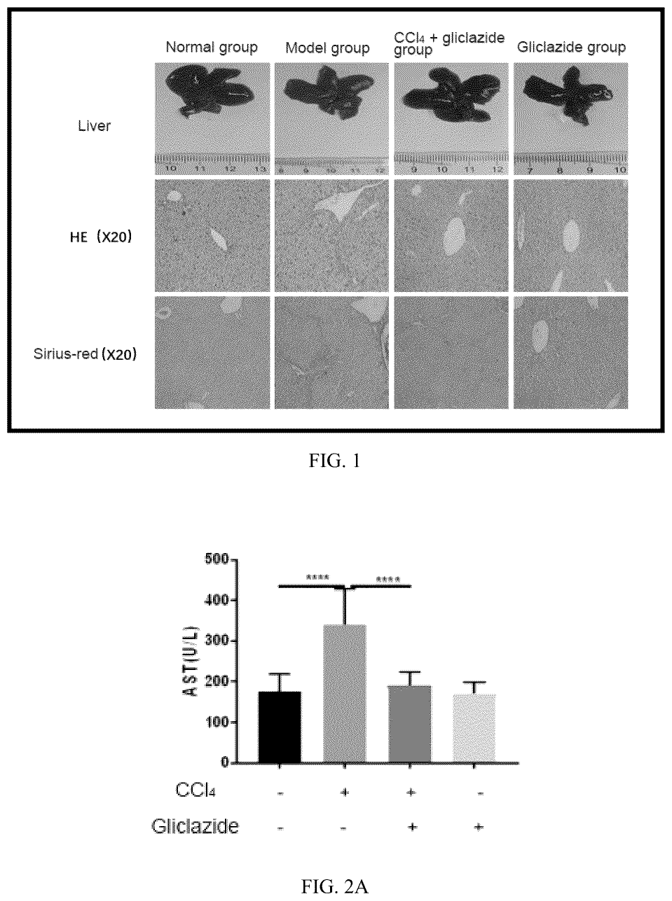

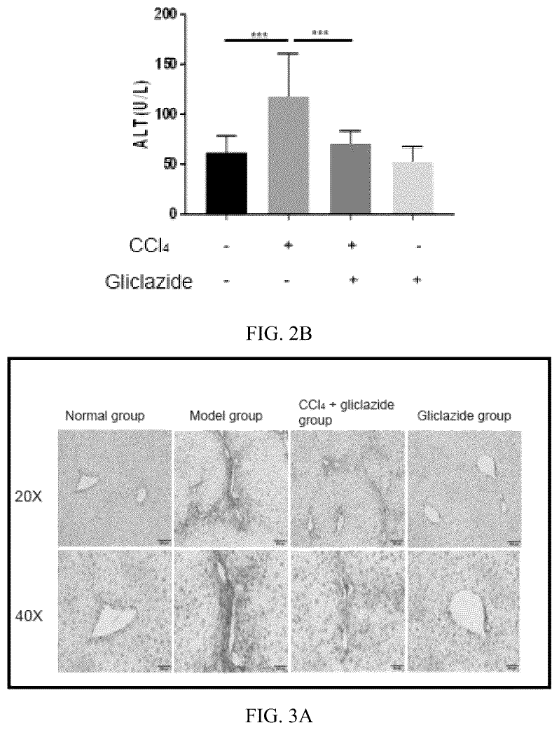

Experimental results are shown in FIG. 1 and FIGS. 2A-2B. The detected levels of ALT and AST of the eyeball blood taken in the model group at the 8th week are significantly increased. Results of the HE staining indicate that in the normal group (i.e., normal control group), the structure of hepatic tissue is clear, the structure of hepatic lobule is normal, the hepatocytes are complete and the cords are clear; in the model group, the hepatic tissue is damaged and the fibrous tissue is significantly proliferated; in the CCl₄+gliclazide group, degrees of hepatic tissue injury and hepatic fibrosis are significantly decreased. Results of Sirius-red staining indicate that in the model group, red collagen fibers are thickened and lengthened, and even form fake lobules in some parts, indicating that the model of mouse hepatic fibrosis is successfully established; in the gliclazide group, no obvious collagen fibers are observed, indicating that gliclazide with the concentration of 500 μM cannot cause hepatic fibrosis in mice.

### Embodiment 2: Protein Expression Level of a Hepatic Fibrosis Index Alpha-Smooth Muscle Actin (α-SMA) In Different Groups Detected By Immunohistochemistry

Experimental materials include C57 mice, primary and secondary antibodies of α-SMA. an immunohistochemistry kit, phosphate buffered saline (PBS) solution, sodium citrate solution and so on.

An experimental method is as follows. The paraffin in the embodiment 1 is used to prepare slices, and immunohistochemical operations are performed in turn according to the following procedures. The slices are sequentially soaked in ethanol with concentrations of 100%, 95%, 80% and 75% for 5 minutes, then rinsed the slices with the PBS solution, catalase is removed by using hydrogen peroxide, and antigens are repaired by sodium citrate repair solution. After cooling, the treated slices are rinsed with the PBS solution, blocked at room temperature for 30 minutes, then incubated overnight at 4° C. with the primary antibodies, and incubated at room temperature with secondary antibodies for 1 hour on the next day. Then, the incubated slices are stained with diaminobenzidine (DAB), counterstained with hematoxylin, differentiated with hydrochloric acid alcohol, dehydrated, blocked, and finally observed and photographed under the microscope.

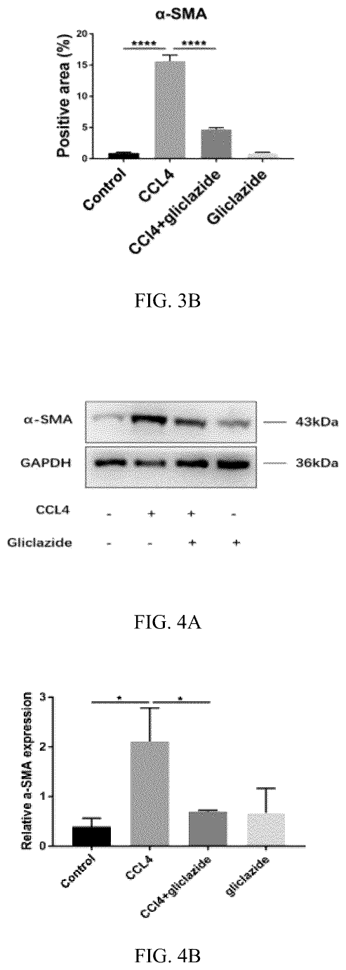

Experimental results are shown in FIGS. 3A-3B, gliclazide can down-regulate expression of α-SMA in CCl₄-induced hepatic fibrosis mice.

### Embodiment 3: Expression of the Hepatic Fibrosis Index α-SMA and Autophagy-Related Indexes Including LC3 and p62 in Different Groups Detected By Western Blotting

Experimental materials include mouse hepatic tissues, a tweezer, a scissor, steel balls with a size of 2 millimeters (mm), a magnet, a cell lysis solution, a PBS solution, a bicinchoninic acid (BCA) protein assay kit, non-fat powdered milk, primary antibodies and corresponding secondary antibodies related to α-SMA, LC3, and p62.

An experimental method is as follows. The hepatic tissue in the embodiment 1 is taken about the size of a grain of rice by using the tweezer and the scissor, added to labeled 1.5 mL Eppendorf (EP) tubes, two steel balls with the size of 2 mm and 200-250 microliter (μL) cell lysis solution are added into each of the EP tubes, after the parameters are set, and the EP tubes are placed in the grinder for full grinding. Then, the steel balls are sucked out by using the magnet, and after being applied to the ice for 30 minutes, the EP tubes are centrifuged at 12000 revolutions per minute (rpm) for 30 minutes at 4° C. A standard curve is made, 1 μL protein samples, 19 μL PBS solution and 200 μL (50:1) BCA working solution are added in each well of a 96-well plate, and two replicate wells are added per well. The 96-well plate is put in a 37° C. drying oven for 30 minutes. The optical density (OD) value at 560 nanometers (nm) is measured by using an enzyme-linked immunosorbent assay (ELISA) on the machine to calculate the concentration of the protein. The protein is boiled in boiling water for 5 minutes before samples loading, and the samples are performed with gel electrophoresis, membrane transfer and blocking sequentially, then the treated samples are exposed after incubating with the primary antibodies and the secondary antibodies.

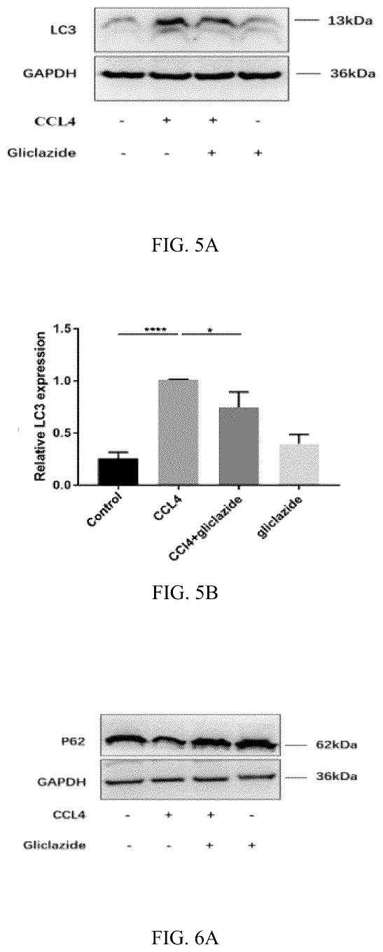

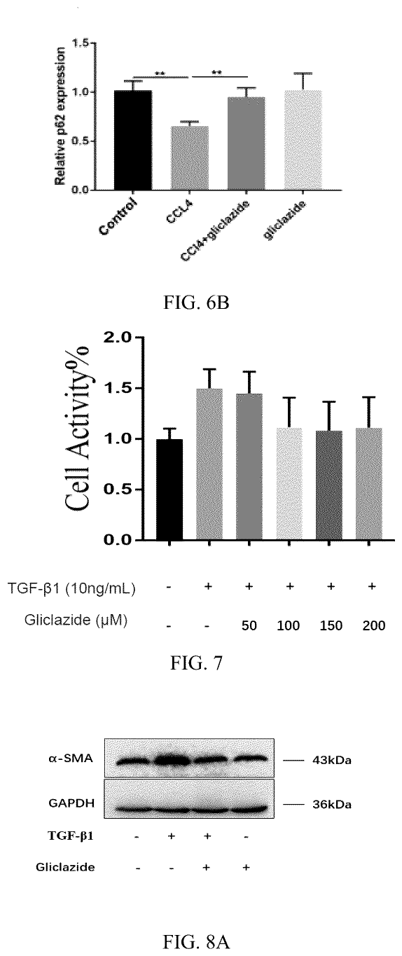

Experimental results are shown in FIGS. 4A-4B to FIGS. 6A-6B, gliclazide significantly decreases the expressions of α-SMA and LC3, and the gliclazide significantly increases the expression level of autophagy substrate p62 in the hepatic tissue.

### Embodiment 4: Effects of Co-Culture of Gliclazide and TGF-β1 on HSCs Proliferation Detected by Cell Counting Kit-8 (CCK8)

Experimental materials include HSCs-T6, a Dulbecco's modified eagle medium (DMEM) complete culture medium, a 96-well plate, a counting chamber (also referred to as hemocytometer), CCK-8 working solution, and the like.

An experimental method is as follows. First, HSCs-T6 are cultured in the DMEM culture medium containing 10% fetal bovine serum, 1% double antibody (penicillin streptomycin solution) and 1% nonessential amino acid (NEAA), and the culture conditions include 37° C. and 5% CO₂. The cells are digested by using trypsin for passage cultivation after the cells are converged to 80% to 90%. Next, the cells are inoculated in the plate, specifically, a density of 7×10⁶ cells per liter (cells/L) cell suspension with a volume of 100 μL is added into each well of the 96-well plate, each group has 6 replicate wells. This experiment is divided into a control group and a TGF-β1 group, TGF-β1+gliclazide (the gliclazide used in this group has different concentrations of 50μM, 100μM, 150μM, and 200μM) medicament groups. After cell attachment, except for the control group and the TGF-β1 group, the other groups are added with gliclazide with the above concentrations respectively, and after 3 hours, TGF-β1 (10 ng/mL) is added to corresponding groups for stimulating HSCs of mice. After 48 hours of incubation, 100 μL mixed solution of “10% CCK8+90% culture medium” is added into each well. Then the incubation is performed at 37° C. for 1˜4 h, a wavelength is set at 450 nm, and an automatic enzyme analyzer is used to read and record absorbance values at the corresponding wavelength. The effects of gliclazide on cell proliferation are detected by CCK8, and the optimal action time and optimal medicament concentration are screened.

Experimental results are shown in FIG. 7, compared with normal values, the stimulation of the TGF-β1 can significantly decrease the proliferation of HSC-T6, the co-culture of gliclazide with concentrations of 100 μM, 150 μM, and 200 μM and the TGF-β1 inhibits the proliferation induced by the TGF-β1, the differences of results in FIG. 7 are statistically significant (P<0.05)

### Embodiment 5: Expression of α-SMA and Autophagy-Related Indexes (LC3 and p62) in HSCs Detected by Western Blotting

Experimental materials include HSCs-T6, a DMEM complete culture medium, cell culture dishes, a cell lysis solution, a PBS solution, a BCA protein assay kit, non-fat powdered milk, primary antibodies and corresponding secondary antibodies related to α-SMA, LC3, and p62.

Experimental method is as follows. First, this experiment is divided into a normal group, a TGF-β1 group, a gliclazide +TGF-β1 group, and a gliclazide group. The concentration of the TGF-β1 is 10 ng/ml, and the concentration of the gliclazide is 100 βM. The the HSCs-T6 is cultured by using the cell culture dish, and inoculated, administrated, added with TGF-β1 according to the conditions described in the embodiment 4. After 48 hours, the incubated cells are rinsed by the precooled PBS solution for 3-4 times, and residual PBS liquid is absorbed with a filter paper, 50 μL radioimmune precipitation assay (RIPA) buffer and 0.5 μL (100:1) phenylmethanesulfonyl fluoride (PMSF) with a concentration of 100millimoles per liter (mmol/L) are added in a 50 mL culture flask and placed on ice for 30 minutes. Then, the protein is scraped by a cell scraper from the culture flask and suctioned out into the EP tubes, and centrifuged at 12000 rpm for 30 minutes at 4° C. A standard curve is made, 1 μL protein samples, 19 μL PBS solution and 200 μL (50:1) BCA working solution are added in each well of the 96-well plate, and two replicate wells are added per well. The 96-well plate is put in a 37° C. drying oven for 30 minutes. The OD value at 560 nm is measured by using ELISA on the machine to calculate the concentration of the protein. The protein is boiled in boiling water for 5 minutes before samples loading, and the samples are performed with gel electrophoresis, membrane transfer and blocking sequentially, then the treated samples are exposed after incubating with the primary antibodies and the secondary antibodies.

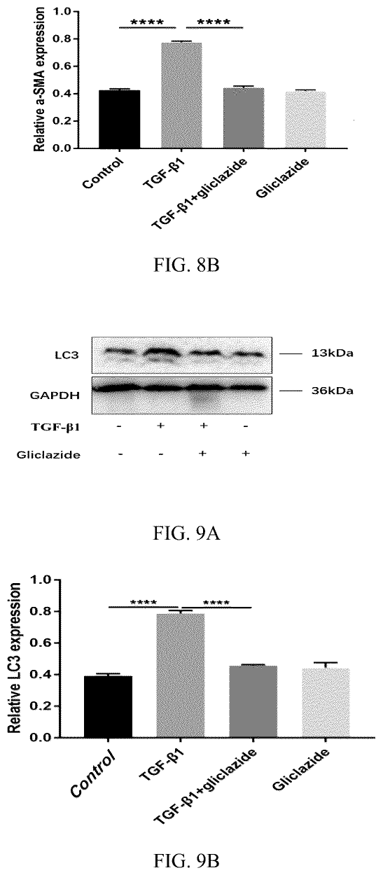

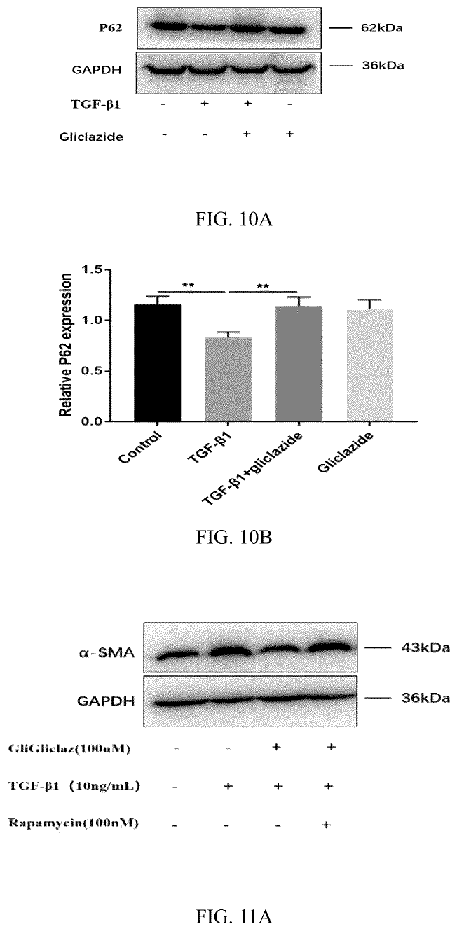

Experimental results are shown in FIGS. 8A-8B to FIGS. 10A-10B, the gliclazide significantly decreases the expressions of α-SMA and LC3, and the gliclazide significantly increases the expression level of autophagy substrate p62 in HSCs.

### Embodiment 6: Expression of α-SMA in HSCs added with an autophagy agonist (rapamycin) by Western blotting

Experimental materials include HSCs-T6, rapamycin, a DMEM complete culture medium, cell culture dishes, a cell lysis solution, a PBS solution, a BCA protein assay kit, non-fat powdered milk, primary antibodies and corresponding secondary antibodies related to α-SMA, LC3, and p62.

Experimental method is as follows. First, this experiment is divided into a normal group, a TGF-β1 group, a gliclazide +TGF-β1 group, and a gliclazide +TGF-β1 +rapamycin group. The concentration of the TGF-β1 is 10 ng/ml, the concentration of the gliclazide is 100 βM, and the concentration of the rapamycin is 100 nanomoles per liter (nM). The TGF-β1 is added at the same time as that in the embodiment 5, and rapamycin and gliclazide are added simultaneously. After 48 hours of incubation, the above groups are detected by the BCA protein assay kit and Western blotting.

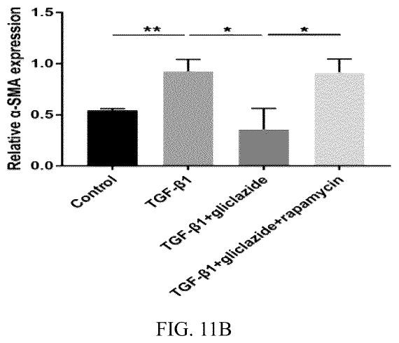

Experimental results are shown in FIGS. 11A-11B, the protein expression level of α-SMA in the co-culture group (i.e., the gliclazide +TGF-ß1 group) is significantly lower than that in the TGF-β1 group, while the protein expression level of α-SMA is significantly higher than that in the other co-culture group (i.e., the gliclazide +TGF-β1 +rapamycin group). The differences of these groups are statistically significant (P<0.05).

The above described embodiments are only some embodiments of the disclosure and do not limit the scope of the disclosure. Without departing from the design spirit of the disclosure, various modifications and improvements made by those skilled in the art to the technical solutions of the disclosure should fall within the scope of protection defined in the claims of the disclosure.

What is claimed is:

1. An application method of an ion channel blocker, comprising: preparing a medicament to treat and prevent hepatic fibrosis by using the ion channel blocker.

2. The application method as claimed in claim 1, wherein the ion channel blocker is a potassium ion channel blocker.

3. The application method as claimed in claim 2, wherein the potassium ion channel blocker comprises gliclazide.

4. The application method as claimed in claim 1, wherein the hepatic fibrosis comprises carbon tetrachloride (CCl₄)-induced hepatic fibrosis.

5. The application method as claimed in claim 3, wherein the gliclazide is applied to prepare a medicament for inhibiting autophagy of hepatic tissue.

6. The application method as claimed in claim 3, wherein the gliclazide is applied to prepare a medicament for inhibiting proliferation of hepatic stellate cells (HSCs).

7. The application method as claimed in claim 6, wherein the HSCs comprise rat hepatic stellate cells (HSCs-T6).

8. The application method as claimed in claim 3, wherein the gliclazide is applied to down-regulate alpha-smooth muscle actin (α-SMA) of hepatocytes.

9. The application method as claimed in claim 3, wherein the gliclazide is applied to prepare a medicament for down-regulating microtubule-associated protein 1A/1B-light chain 3 (LC3) of hepatocytes.

10. The application method as claimed in claim 3, wherein the gliclazide is applied to prepare a medicament for up-regulating sequestosome 1 (p62) of hepatocytes.
